(19) United States
(12) Patent Application Publication
(10) Pub. No.: US 2008/0102091 A1
(43) Pub. Date: May 1, 2008

(54) Vaccines

(75) Inventors: Joseph D. Cohen, Rixensart (BE); Nadia Gabriela Tornieporth, Rixensart (BE)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GLAXOSMITHKLINE BIOLOGICALS SA, Rixensart (BE)

(21) Appl. No.: 11/575,414

(22) PCT Filed: Sep. 14, 2005
(86) PCT No.: PCT/EP05/09995

§ 371 Date: Mar. 16, 2007

(30) Foreign Application Priority Data
Sep. 16, 2004 (GB) 0420634.8

(51) Int. Cl. A61K 39/15; A61P 33/06

(52) U.S. Cl. 424/272.1

(57) ABSTRACT

The present invention relates to a novel use of a malaria antigen to immunise against malarial disease. The invention relates in particular to the use of sporozoite antigens, in particular circumsporozoite (CS) protein or fragments thereof, to immunise against severe malarial disease.

The present invention relates to a novel use of a malaria antigen to immunise against malarial disease. The invention relates in particular to the use of sporozoite antigens, in particular circumsporozoite (CS) protein or fragments thereof, to immunise against severe malarial disease.

Malaria is one of the world's major health problems. During the 20th century, economic and social development, together with anti malarial campaigns, have resulted in the eradication of malaria from large areas of the world, reducing the affected area of the world surface from 50% to 27%. Nonetheless, given expected population growth it is projected that by 2010 half of the world's population, nearly 3.5 billion people, will be living in areas where malaria is transmitted¹. Current estimates suggest that there are well in excess of 1 million deaths due to malaria every year, and the staggering economic costs for Africa alone are equivalent to US$ 100 billion annually².

These figures highlight the global malaria crisis and the challenges it poses to the international health community. The reasons for this crisis are multiple and range from the emergence of widespread resistance to available, affordable and previously highly effective drugs, to the breakdown and inadequacy of health systems to the lack of resources. Unless ways are found to control this disease, global efforts to improve health and child survival, reduce poverty, increase security and strengthen the most vulnerable societies will fail.

One of the most acute forms of the disease is caused by the protozoan parasite Plasmodium falciparum which is responsible for most of the mortality attributable to malaria.

The life cycle of P. falciparum is complex, requiring two hosts, man and mosquito for completion. The infection of man is initiated by the inoculation of sporozoites in the saliva of an infected mosquito. The sporozoites migrate to the liver and there infect hepatocytes (liver stage) where they differentiate, via the exoerythrocytic intracellular stage, into the merozoite stage which infects red blood cells (RBC) to initiate cyclical replication in the asexual blood stage. The cycle is completed by the differentiation of a number of merozoites in the RBC into sexual stage gametocytes which are ingested by the mosquito, where they develop through a series of stages in the midgut to produce sporozoites which migrate to the salivary gland.

The sporozoite stage of P. falciparum has been identified as one potential target of a malaria vaccine. The major surface protein of the sporozoite is known as circumsporozoite protein (CS protein). This protein has been cloned, expressed and sequenced for a variety of strains for example the NF54 strain, clone 3D7 (Caspers et al., Mol. Biochem. Parasitol. 35, 185-190, 1989). The protein from strain 3D7 is characterised by having a central immunodominant repeat region comprising a tetrapeptide Asn-Ala-Asn-Pro repeated 40 times but interspersed with four minor repeats Asn-Val-Asp-Pro. In other strains the number of major and minor repeats varies as well as their relative position. This central portion is flanked by an N and C terminal portion composed of non-repetitive amino acid sequences designated as the repeatless portion of the CS protein.

GlaxoSmithKline Biologicals' RTS,S malaria vaccine based on CS protein has been under development since 1987 and is currently the most advanced malaria vaccine candidate being studied⁴. This vaccine specifically targets the pre-erythrocytic stage of P. falciparum, and confers protection against infection by P. falciparum sporozoites delivered via laboratory-reared infected mosquitoes in malaria-naïve adult volunteers, and against natural exposure in semi-immune adults^{5,6}.

RTS,S/AS02A (RTS,S plus adjuvant) was used in consecutive Phase I studies undertaken in The Gambia involving children aged 6-11 and 1-5 years, which confirmed that the vaccine was safe, well-tolerated and immunogenic⁷. Subsequently a paediatric vaccine dose was selected and studied in a phase I study involving Mozambican children aged 1-4 years where it was found to be safe, well tolerated and immunogenic⁸.

However, it is a long held notion that to achieve protection from clinical disease caused by P. falciparum in conditions of natural exposure would require more than a single antigen, and would require multiple antigens representing multiple stages of the parasite life cycle (Page: 3

Webster, Daniel and Hill, Adrian V. S. Progress with new malaria vaccines. Bull World Health Organ, December 2003, vol. 81, no. 12, p. 902-909. ISSN 0042-9686; Hoffman S. Save the children. Nature. 2004 Aug. 19;430(7002):940-1). It has also been a generally held concept that an antigen such as CS from the pre-erythrocytic stages of the parasite would not be the preferred antigen to provide protection against severe disease, since severe disease is caused by asexual stage parasites and pre-erythrocytic antigens such as CS are not expressed on asexual stage parasites.

Surprising results have now been obtained with a pre-erythrocytic malaria antigen in a trial in young African children. It has been discovered that the CS protein based RTS,S vaccine can confer not only protection against infection under natural exposure but also protection against a wide spectrum of clinical illness caused by P. falciparum. Children who received the RTS,S vaccine experienced fewer serious adverse events, hospitalisations, and severe complications from malaria, including death, than did those in the control group.

In particular, the finding that the incidence of severe malaria disease could be reduced by this CS based vaccine was unexpected and surprising. Severe malaria disease is described in the WHO guide to clinical practice (Page: 3

World Health Organization. Management of severe malaria, a practical handbook. Second edition, 2000. http://mosquito.who.int/docs/hbsm.pdf). Classification of children according to the WHO-based definition for severe malaria identifies children who are very sick and at high risk of dying. High risk may be taken to mean about a 30% or greater risk dying.

Furthermore, the RTS,S vaccine efficacy against both new infections or clinical episodes appears either not to wane or to do so slowly. At the end of the 6 months follow up in the trial, the vaccine remained efficacious as there was a significant difference in the prevalence of infection. This is in sharp contrast from previous trials in malaria naïve volunteers or Gambian adults which suggested that vaccine efficacy was short lived^{6,23}.

Therefore the present invention provides the use of a Plasmodium antigen which is expressed at the pre-erythrocytic stage, preferably a sporozoite antigen, in the manufacture of a medicament for vaccinating against severe malaria disease, in combination with a pharmaceutically acceptable adjuvant or carrier.

The invention is particularly concerned with reducing the incidence of severe P. falciparum disease.

The preferred target population for such a vaccine is children, in particular children under 5 years of age and especially children 1-4 years of age.

Preferably the Plasmodium antigen is a P. falciparum antigen.

The antigen may be selected from any antigen which is expressed on the sporozoite or other pre-erythrocytic stage of the parasite such as the liver stage. Preferably the antigen is selected from circumsporozoite (CS) protein, liver stage antigen-1 (LSA-1), liver stage antigen-3 (LSA-3), thrombospondin related anonymous protein (TRAP) and apical merezoite antigen-1 (AMA-1) which has recently been show to be present at the liver stage (in addition to the erythrocytic stage). All of these antigens are well known in the field. The antigen may be the entire protein or an immunogenic fragment thereof. Immunogenic fragments of malaria antigens are well know, for example the ectodomain from AMA-1.

Preferably the Plasmodium antigen is fused to the surface antigen from hepatitis B (HBsAg).

A preferred antigen for use in the invention is derived from the circumsporozoite (CS) protein and is preferably in the form of a hybrid protein with HBsAg. The antigen may be the entire CS protein or part thereof, including a fragment or fragments of the CS protein which fragments may be fused together.

Preferably the CS protein based antigen is in the form of a hybrid protein comprising substantially all the C-terminal portion of the CS protein of Plasmodium, four or more tandem repeats of the CS protein immunodominant region, and the surface antigen from hepatitis B (HBsAg). Preferably the hybrid protein comprises a sequence which contains at least 160 amino acids which is substantially homologous to the C-terminal portion of the CS protein. In particular “substantially all” the C terminal portion of the CS protein includes the C terminus devoid of the hydrophobic anchor sequence. The CS protein may be devoid of the last 12 amino-acids from the C terminal.

Most preferably the hybrid protein for use in the invention is a protein which comprises a portion of the CS protein of P. falciparum substantially as corresponding to amino acids 207-395 of P. falciparum 3D7 clone, derived from the strain NF54 (Caspers et al, supra) fused in frame via a linear linker to the N-terminal of HBsAg. The linker may comprise a portion of preS2 from HBsAg.

Preferred CS constructs for use in the present invention are as outlined in WO 93/10152. Most preferred is the hybrid protein known as RTS as described in WO 93/10152 (wherein it is denoted RTS*) and WO 98/05355, the whole contents of both of which are incorporated herein by reference.

A particularly preferred hybrid protein is the hybrid protein known as RTS which consists of:

A metbionine-residue, encoded by nucleotides 1059 to 1061, derived from the Sacchromyes cerevisiae TDH3 gene sequence. (Musti A. m. et al Gene 1983 25 133-143).

Three amino acids, Met Ala Pro, derived from a nucleotide sequence (1062 to 1070) created by the cloning procedure used to construct the hybrid gene.

A stretch of 189 amino acids, encoded by nucleotides 1071 to 1637 representing amino acids 207 to 395 of the circumsporozoite protein (CSP) of Plasmodium falciparum strain 3D7 (Caspers et al, supra).

An amino acid (Gly) encoded by nucleotides 1638 to 1640, created by the cloning procedure used to construct the hybrid gene.

Four amino acids, Pro Val Thr Asn, encoded by nucleotides 1641 to 1652, and representing the four carboxy terminal residues of the hepatitis B virus (adw serotype) preS2 protein (Nature 280: 815-819, 1979).

A stretch of 226 amino acids, encoded by nucleotides 1653 to 2330, and specifying the S protein of hepatitis B virus (adw serotype).

Preferably the RTS is in the form of mixed particles RTS,S.

The preferred RTS,S construct comprises two polypeptides RTS and S that are synthesized simultaneously and during purification spontaneously form composite particulate structures (RTS,S).

The RTS protein is preferably expressed in yeast, most preferably S. cerevisiae. In such a host, RTS will be expressed as lipoprotein particle. The preferred recipient yeast strain preferably already carries in its genome several integrated copies of an hepatitis B S expression cassette. The resulting strain synthesizes therefore two polypeptides, S and RTS, that spontaneously co-assemble into mixed (RTS,S) lipoprotein particles. These particles, advantageously present the CSP sequences of the hybrid at their surface. Advantageously the ratio of RTS: S in these mixed particles is 1:4.

The invention allows the use of a single malaria antigen in a vaccine, contrary to what was previously thought would be required for the generation of protection, in particular protection against severe disease. In accordance with the invention therefore, the RTS or other antigen is preferably the sole malaria antigen in the vaccine.

In another aspect, the invention provides the use of an antigen from a single malarial protein in the manufacture of a medicament for use in vaccination against severe malaria. The malarial protein may be any of the proteins described herein including CS protein, AMA-1, TRAP, LSA-1 and LSA-3. Most preferably it is CS protein, in hybrid form as described herein.

The invention further provides a method of preventing or reducing severe malaria which method comprises administering to a subject a composition comprising a malaria antigen which is expressed at the pre-erythrocytic stage and an adjuvant. The antigens and adjuvants are as described herein. The preferred subjects are children, preferably in the age ranges described herein.

A suitable vaccination schedule for use in the invention includes the administration of 3 doses of vaccine, at one month intervals.

Severe malaria may be defined according to the WHO guidelines for clinical practice (supra). In the study described herein the criteria for defining severe malaria were derived from the WHO guide to clinical practice and are given in the table below.

As the primary endpoint, clinical episodes of malaria defined in the study were required to have the presence of P. falciparum asexual parasitemia>15 000 per μL on Giemsa stained thick blood films and the presence of fever (axillary temperature>37.5° C.) ≧37.5° C.

The definition for severe malaria was the additional presence of one or more of the following: severe malaria anaemia (PCV <15%), cerebral malaria (Blantyre coma score<2) or severe disease of other body systems which could include multiple seizures (two or more generalized convulsions in the previous 24 hours), prostration (defined as inability to sit unaided), hypoglycaemia<2.2 mmol/dL or <40 mg/dL), clinically suspected acidosis or circulatory collapse. These are given in Table 1 below.

Severe malaria case definition

| | | |
|---|---|---|
| Severe malaria | Asexual parasitemia | |
| anemia | definitive reading | |
| | Hematocrit < 15% | |
| | No other more probable | |
| | cause of illness | |
| Cerebral | Asexual parasitemia | Assess coma score after |
| malaria | definitive reading | correction of hypoglycemia |
| | Coma score ≦ 2 | and 60 minutes after control |
| | No other identifiable | of fits. If fitting cannot be |
| | cause of loss of | controlled within 30 minutes |
| | consciousness | child is included |
| Severe malaria | Asexual parasitemia | |
| (other) | definitive reading | |
| | No other more probable | |
| | cause of illness | |
| | Does not meet criteria for | |
| | severe malaria anemia or | |
| | cerebral malaria | |
| | One of the following: | |
| | Multiple seizures | Two or more generalized |
| | | convulsions within a 24-hour |
| | | period prior to admission |
| | Prostration | Inability to sit unaided |
| | Hypoglycemia | <2.2 mmol/dL or <40 mg/dL |
| | Acidosis | Document supportive signs |
| | | and/or laboratory readouts |
| | Circulatory collapse | Document supportive signs |
| | | and/or laboratory readouts |

In accordance with the invention, an aqueous solution of the purified hybrid protein may be used directly and combined with a suitable adjuvant or carrier. Alternatively, the protein can be lyophilized prior to mixing with a suitable adjuvant or carrier.

The preferred vaccine dose in accordance with the invention is between 1-100 μg RTS,S per dose, more preferably 5 to 75 μg RTS,S, most preferably a dose of 25 μg RTS,S protein, preferably in 250 μl (final liquid formulation). This is the preferred dose for use in children, in particular children below five years of age and more particularly children aged 1-4, and represents one half of the preferred adult dose. The preferred adult dose is between 1-100 μg RTS,S per dose, more preferably 5 to 75 μg RTS,S, most preferably a dose of 50 μg RTS,S in 500 μl (final liquid formulation).

In accordance with the invention the antigen is combined with an adjuvant or carrier. Preferably an adjuvant is present, in particular an adjuvant which is a preferential stimulator of a Th1 type response.

Suitable adjuvants include but not limited to, detoxified lipid A from any source and non-toxic derivatives of lipid A, saponins and other immunostimulants which are preferential stimulators of a Th1 cell response (also herein called a Th1 type response).

An immune response may be broadly divided into two extreme categories, being a humoral or cell mediated immune response (traditionally characterised by antibody and cellular effector mechanisms of protection respectively). These categories of response have been termed TH1-type responses (cell-mediated response), and TH2-type immune responses (humoral response).

Extreme TH1-type immune responses may be characterised by the generation of antigen specific, haplotype restricted cytotoxic T lymphocytes, and natural killer cell responses. In mice TH1-type responses are often characterised by the generation of antibodies of the IgG2a subtype, whilst in the human these correspond to IgG1 type antibodies. TH2-type immune responses are characterised by the generation of a range of immunoglobulin isotypes including in mice IgG1.

It can be considered that the driving force behind the development of these two types of immune responses are cytokines. High levels of TH1-type cytokines tend to favour the induction of cell mediated immune responses to the given antigen, whilst high levels of TH2-type cytokines tend to favour the induction of humoral immune responses to the antigen. The distinction of TH1and TH2-type immune responses is not absolute, and can take the form of a continuum between these two extremes. In reality an individual will support an immune response which is described as being predominantly TH1or predominantly TH2. However, it is often convenient to consider the families of cytokines in terms of that described in murine CD4+ve T cell clones by Mosmann and Coffman (Mosmann, T. R. and Coffman, R. L. (1989) TH1 and TH2 cells: different patterns of lymphokine secretion lead to different functional properties. Annual Review of Immunology, 7, p145-173). Traditionally, TH1-type responses are associated with the production of the INF-γ cytokines by T-lymphocytes. Other cytokines often directly associated with the induction of TH1-type immune responses are not produced by T-cells, such as IL-12. In contrast, TH2- type responses are associated with the secretion of IL-4, IL-5, IL-6, IL-10 and tumour necrosis factor-β(TNF-β).

It is known that certain vaccine adjuvants are particularly suited to the stimulation of either TH1 or TH2-type cytokine responses. Traditionally indicators of the TH1:TH2 balance of the immune response after a vaccination or infection includes direct measurement of the production of TH1 or TH2 cytokines by T lymphocytes in vitro after restimulation with antigen, and/or the measurement (at least in mice) of the IgG1:IgG2a ratio of antigen specific antibody responses.

Thus, a TH1-type adjuvant is one which stimulates isolated T-cell populations to produce high levels of TH1-type cytokines when re-stimulated with antigen ill vitro, and induces antigen specific immunoglobulin responses associated with TH1-type isotype.

Adjuvants which are capable of preferential stimulation of the TH1 cell response are described in WO 94/00153 and WO 95/17209.

Preferred Th1-type immunostimulants which may be formulated to produce adjuvants suitable for use in the present invention include and are not restricted to the following.

It has long been known that enterobacterial lipopolysaccharide (LPS) is a potent stimulator of the immune system, although its use in adjuvants has been curtailed by its toxic effects. A non-toxic derivative of LPS, monophosphoryl lipid A (MPL), produced by removal of the core carbohydrate group and the phosphate from the reducing-end glucosamine, has been described by Ribi et al (1986, Immunology and Immunopharmacology of bacterial endotoxins, Plenum Publ. Corp., N.Y., p 407-419) and has the following structure:

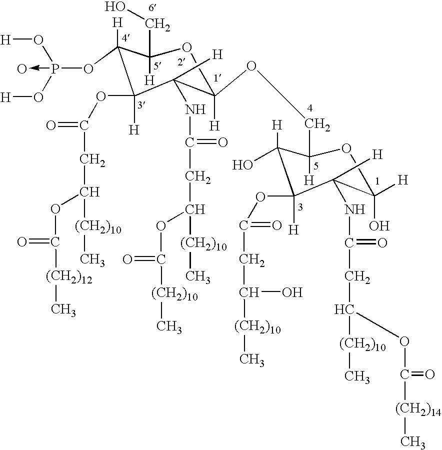

A further detoxified version of MPL results from the removal of the acyl chain from the 3-position of the disaccharide backbone, and is called 3-O-Deacylated monophosphoryl lipid A (3D-MPL). It can be purified and prepared by the methods taught in GB 2122204B, which reference also discloses the preparation of diphosphoryl lipid A, and 3-O-deacylated variants thereof.

A preferred form of 3D-MPL is in the form of an emulsion having a small particle size less than 0.2 μm in diameter, and its method of manufacture is disclosed in WO 94/21292. Aqueous formulations comprising monophosphoryl lipid A and a surfactant have been described in WO9843670.

The bacterial lipopolysaccharide derived adjuvants to be used in the present invention may be purified and processed from bacterial sources, or alternatively they may be synthetic. For example, purified monophosphoryl lipid A is described in Ribi et al 1986 (supra), and 3-O-Deacylated monophosphoryl or diphosphoryl lipid A derived from Salmonella sp. is described in GB 2220211 and U.S. Pat. No. 4,912,094. Other purified and synthetic lipopolysaccharides have been described (Hilgers et al., 1986, Int. Arch. Allergy. Immunol., 79(4):392-6; Hilgers et al., 1987, Immunology, 60(1):141-6; and EP 0 549 074 B1). A particularly preferred bacterial lipopolysaccharide adjuvant is 3D-MPL.

Accordingly, the LPS derivatives that may be used in the present invention are those immunostimulants that are similar in structure to that of LPS or MPL or 3D-MPL. In another alternative the LPS derivatives may be an acylated monosaccharide, which is a sub-portion to the above structure of MPL.

Saponins are also preferred Th1 immunostimulants in accordance with the invention. Saponins are well known adjuvants and are taught in: Lacaille-Dubois, M and Wagner H. (1996. A review of the biological and pharmacological activities of saponins. Phytomedicine vol 2 pp 363-386). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B1. The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1. Also described in these references is the use of QS7 (a non-haemolytic fraction of Quil-A) which acts as a potent adjuvant for systemic vaccines. Use of QS21 is further described in Kensil et al. (1991. J. Immunology vol 146, 431-437). Combinations of QS21 and polysorbate or cyclodextrin are also known (WO 99/10008). Particulate adjuvant systems comprising fractions of QuilA, such as QS21 and QS7 are described in WO 96/33739 and WO 96/11711.

Another preferred immunostimulant is an immunostimulatory oligonucleotide containing unmethylated CpG dinucleotides (“CpG”). CpG is an abbreviation for cytosine-guanosine dinucleotide motifs present in DNA. CpG is known in the art as being an adjuvant when administered by both systemic and mucosal routes (WO 96/02555, EP 468520, Davis et al., J. Immunol, 1998, 160(2):870-876; McCluskie and Davis, J. Immunol., 1998, 161(9):4463-6). Historically, it was observed that the DNA fraction of BCG could exert an anti-tumour effect. In further studies, synthetic oligonucleotides derived from BCG gene sequences were shown to be capable of inducing immunostimulatory effects (both in vitro and in vivo). The authors of these studies concluded that certain palindromic sequences, including a central CG motif, carried this activity. The central role of the CG motif in immunostimulation was later elucidated in a publication by Krieg, Nature 374, p 546 1995. Detailed analysis has shown that the CG motif has to be in a certain sequence context, and that such sequences are common in bacterial DNA but are rare in vertebrate DNA. The immunostimulatory sequence is often: Purine, Purine, C, G, pyrimidine, pyrimidine; wherein the CG motif is not methylated, but other unmethylated CpG sequences are known to be immunostimulatory and may be used in the present invention.

In certain combinations of the six nucleotides a palindromic sequence is present. Several of these motifs, either as repeats of one motif or a combination of different motifs, can be present in the same oligonucleotide. The presence of one or more of these immunostimulatory sequences containing oligonucleotides can activate various immune subsets, including natural killer cells (which produce interferon y and have cytolytic activity) and macrophages (Wooldrige et al Vol 89 (no. 8), 1977). Other unmethylated CpG containing sequences not having this consensus sequence have also now been shown to be immunomodulatory.

CpG when formulated into vaccines, is generally administered in free solution together with free antigen (WO 96/02555; McCluskie and Davis, supra) or covalently conjugated to an antigen (WO 98/16247), or formulated with a carrier such as aluminium hydroxide ((Hepatitis surface antigen) Davis et al. supra ; Brazolot-Millan et al., Proc. Natl. Acad. Sci.,USA, 1998, 95(26), 15553-8).

Such immunostimulants as described above may be formulated together with carriers, such as for example liposomes, oil in water emulsions, and or metallic salts, including aluminium salts (such as aluminium hydroxide). For example, 3D-MPL may be formulated with aluminium hydroxide (EP 0 689 454) or oil in water emulsions (WO 95/17210); QS21 may be advantageously formulated with cholesterol containing liposomes (WO 96/33739), oil in water emulsions (WO 95/17210) or alum (WO 98/15287); CpG may be formulated with alum (Davis et al. supra ; Brazolot-Millan supra) or with other cationic carriers.

Combinations of immunostimulants are also preferred, in particular a combination of a monophosphoryl lipid A and a saponin derivative (WO 94/00153; WO 95/17210; WO 96/33739; WO 98/56414; WO 99/12565; WO 99/11241), more particularly the combination of QS21 and 3D-MPL as disclosed in WO 94/00153. Alternatively, a combination of CpG plus a saponin such as QS21 also forms a potent adjuvant for use in the present invention.

Thus, suitable adjuvant systems include, for example, a combination of monophosphoryl lipid A, preferably 3D-MPL, together with an aluminium salt.

An enhanced system involves the combination of a monophosphoryl lipid A and a saponin derivative particularly the combination of QS21 and 3D-MPL as disclosed in WO 94/00153, or a less reactogenic composition where the QS21 is quenched in cholesterol containing liposomes (DQ) as disclosed in WO 96/33739.

A particularly potent adjuvant formulation involving QS21, 3D-MPL & tocopherol in an oil in water emulsion is described in WO 95/17210 and is another preferred formulation for use in the invention.

Another preferred formulation comprises a CpG oligonucleotide alone or together with QS21, 3D-MPL or together with an aluminium salt.

Accordingly in one embodiment of the present invention there is provided the use of detoxified lipid A or a non-toxic derivative of lipid A, more preferably monophosphoryl lipid A or derivative thereof such as 3D-MPL, in combination with a malaria antigen as described herein, for the manufacture of a vaccine for the prevention of severe malaria disease.

Preferably a saponin is additionally used, preferably QS21.

Preferably the invention further employs an oil in water emulsion or liposomes.

Preferred combinations of adjuvants for use in the present invention are:

1. 3D-MPL, QS21 and an oil in water emulsion.

2. 3D-MPL and QS21 in liposome formulation.

3. 3D-MPL, QS21 and CpG in a liposome formulation.

The amount of the protein of the present invention present in each vaccine dose is selected as an amount which induces an immunoprotective response without significant, adverse side effects in typical vaccines. Such amount will vary depending upon which specific immunogen is employed and whether or not the vaccine is adjuvanted. Generally, it is expected that each does will comprise 1-1000 μg of protein, preferably 1-200 μg most preferably 10-100 μg. An optimal amount for a particular vaccine can be ascertained by standard studies involving observation of antibody titres and other responses in subjects. Following an initial vaccination, subjects will preferably receive a boost in about 4 weeks, followed by repeated boosts every six months for as long as a risk of infection exists. Preferred amounts of RTS,S protein are also as given hereinabove.

The vaccines of the invention may be provided by any of a variety of routes such as oral, topical, subcutaneous, mucosal (typically intravaginal), intraveneous, intramuscular, intranasal, sublingual, intradermal and via suppository.

Immunisation can be prophylactic or therapeutic. The invention described herein is primarily but not exclusively concerned with prophylactic vaccination against malaria, more particularly prophylactic vaccination to prevent or to reduce the likelihood of severe malaria disease.

Appropriate pharmaceutically acceptable carriers or excipients for use in the invention are well known in the art and include for example water or buffers. Vaccine preparation is generally described in Pharmaceutical Biotechnology, Vol. 61 Vaccine Design—the subunit and adjuvant approach, edited by Powell and Newman, Plenum Press New York, 1995. New Trends and Developments in Vaccines, edited by Voller et al., University Park Press, Baltimore, Md., U.S.A. 1978. Encapsulation within liposomes is described, for example, by Fullerton, U.S. Pat. No. 4,235,877. Conjugation of proteins to macromolecules is disclosed, for example, by Likhite, U.S. Pat. No. 4,372,945 and by Armor et al., U.S. Pat. No. 4,474,757.

### EXAMPLES

### Materials and Methods

### Study Area

The trial was conducted at the Centro de Investigação em Saude da Manhiça [CISM] (Manhiça Health Research Centre), in Manhiça District (Maputo Province), in southern Mozambique between April 2003 and May 2004. The characteristics of the area have been described in detail elsewhere⁹. The climate is subtropical with two distinct seasons: a warm and rainy season from November to April, and a generally cool and dry season during the rest of the year. During 2003 annual rainfall was 1286 mm. Perennial malaria transmission with marked seasonality is mostly due to P. falciparum. Anopheles funestus is the main vector and the estimated entomologic inoculation rate (EIR) for 2002 was 38. Combination therapy based on amodiaquine and sulphadoxine-pyrimethamine (SP) is the first line treatment for uncomplicated malaria, and is readily available at health facilities. Adjacent to CISM is the Manhiça Health Center, the 10 bed referral health facility. The district health network consists of a further 8 peripheral health posts and a rural Hospital.

### Study Design

The study was a Phase IIb double-blind, randomised and controlled trial to evaluate the safety, immunogenicity and efficacy of GSK Biologicals' RTS,S/AS02A malaria vaccine. The primary objective was to estimate the efficacy against clinical episodes of P. falciparum malaria in children aged 1 to 4 years at first vaccination over a 6 month surveillance period starting 14 days after dose 3.

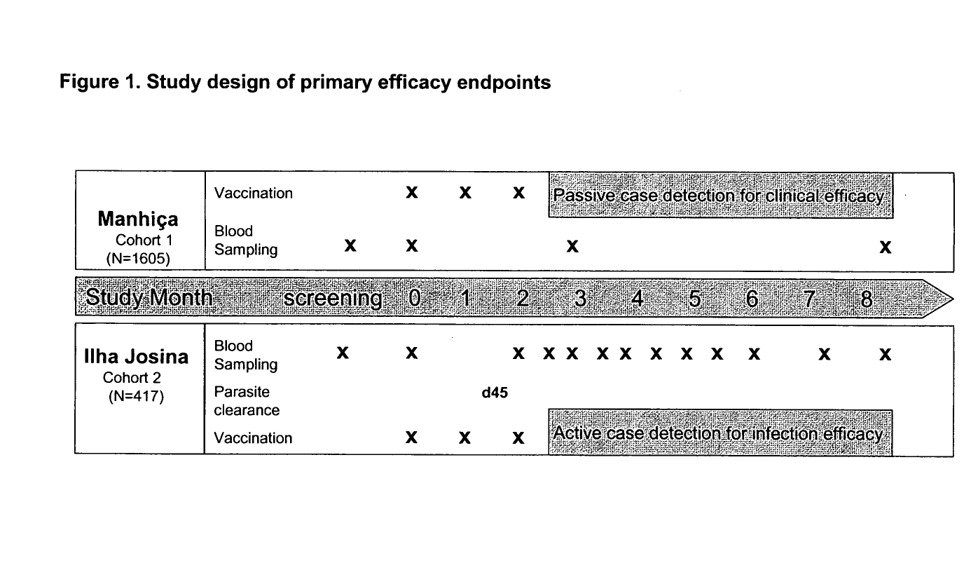

The trial was designed to examine the efficacy of the vaccine at two points in the life cycle and pathogenesis of malaria: infection and clinical disease. These two endpoints were measured simultaneously in two cohorts based at two different sites (FIG. 1). Cohort 1, recruited from an area of 10 Km radius around Manhiça, contributed to the assessment of the primary endpoint of protection against clinical disease determined through passive case detection at the Manhiça Health Center and the Maragra Health Post. Cohort 2 was recruited in Ilha Josina, an agricultural and marshy lowland area 55 km north of Manhiça, and was followed to detect new infections through a combination of active and passive surveillance.

For cohort 1, 704 evaluable subjects per group were needed in order to have 80% power to detect a lower confidence limit of vaccine efficacy of 15%, assuming clinical P. falciparum attack rate over the surveillance period of 11% in the control group and vaccine efficacy of 50%. For cohort 2, 116 evaluable children per group were needed to provide 86% power to detect a vaccine efficacy of 50% in the prevention of new infections with a lower confidence limit of 20% assuming a rate of new infections of 50% over the surveillance period.

The protocol was approved by the National Mozambican Ethics Review Committee, the Hospital Clinic of Barcelona Ethics Review Committee and the Program for Appropriate Technology in Health (PATH) Human Subjects Protection Committee. The trial was conducted according to the ICH Good Clinical Practice guidelines, and was monitored by GlaxoSmithKline Biologicals. A Local Safety Monitor and a Data and Safety Monitoring Board closely reviewed the conduct and results of the trial.

### Screening and Informed Consent

CISM runs a demographic surveillance system in the study area¹⁰. Lists of potentially eligible resident children were produced from this census. They were visited at home, information sheets were read to parents or guardians and criteria for recruitment were checked. These included confirmed residency in the study area and full immunisation with EPI vaccines. Interested parents/guardians were invited to the Manhiça Health Centre or the Ilha Josina Health Post. At first visit, the information sheet was again read and explained to groups of parents/guardians by specially trained staff. Individual consent was sought only after they passed an individual oral comprehension test designed to check understanding of this information. They were then invited to sign (or thumb-print if not literate) the informed consent document. A member of the community acted as an impartial witness and countersigned the consent form. Screening included a brief medical history and examination, blood sampling by fingerprick for haematology and biochemistry tests.

Children were excluded from participation if they had a history of allergic disease, hematocrit <25%, were malnourished (weight for height ≦3 Z score), had clinically significant chronic or acute disease or abnormal haematology or biochemical parameters. Eligible subjects were enrolled in the study starting on the first day of vaccination and given a unique study number and individual photographic identification card.

Randomisation and Immunisation

2022 children aged 1-4 years were recruited and randomised to receive three doses of either RTS,S/AS02A candidate malaria vaccine or a control vaccination regime at Manhiça Health Center or Ilha Josina Health Post. The randomisation was performed at GSK Biologicals using a blocking scheme (1:1 ratio, block size=6).

RTS,S consists of a hybrid molecule recombinantly expressed in yeast, in which the CS protein 10, central tandem repeat and carboxyl-terminal regions are fused N terminal to the S antigen of Hepatitis B virus (HBsAg) in a particle that also includes the unfused S antigen. A full dose of RTS,S/AS02A (GlaxoSmithKline Biologicals, Rixensart, Belgium) contains 50 μg of lyophilised RTS,S antigen reconstituted in 500 μL of AS02A adjuvant (oil in water emulsion containing the immunostimulants 3D-MPL® [Corixa Inc., WA, USA] and QS21, 50 μg of each). A one-half adult dose was used in this trial; i.e. a 250 μL dose volume containing 25 μg of RTS,S antigen in 250 μL AS02 adjuvant (containing 25 μg of each of 3D-MPL and QS21).

Because routine hepatitis B vaccination was introduced into the EPI schedule of Mozambique in July 2001, children aged 12 to 24 months had already received Hepatitis B immunisation. Accordingly, children less than 24 months received as control vaccines two doses of the 7-valent pneumococal conjugate vaccine (Prevnar® Wyeth Lederle Vaccines, New Jersey, USA) at the first and third vaccination and one dose of Haemophilus influanzae type b vaccine (Hiberix™ GlaxoSmithKline Biologicals, Rixensart, Belgium) at the second vaccination. For children older than 24 months, the control vaccine was the paediatric hepatitis B vaccine (Engerix-B® GlaxoSmithKline Biologicals, Rixensart, Belgium). Full doses (0.5 ml dose volume) were given to the control group.

Both RTS,S/AS02A and control vaccines were administered intramuscularly in the deltoid region of alternating arms according to a 0, 1, 2 month vaccination schedule. Since the vaccines used are of distinct appearance and volume, special precautions were taken to ensure the double-blind nature of the trial. A vaccination team prepared the vaccine and masked the contents of the syringe with an opaque tape prior to immunisation. This team was not involved in any other study procedures, including surveillance for endpoints.

### Follow Up for Safety and Reactogenicity

After each vaccination, study participants were observed for at least one hour. Trained field workers visited the children at home every day for the three following days to record any adverse event. Solicited local and general adverse events were documented over this period 12. Unsolicited adverse events were recorded for 30 days after each dose through the hospital morbidity surveillance system. Serious adverse events (SAEs) were detected in a similar way and recorded throughout the study. Study children were visited at home once a month, starting 60 days after dose 3. During the visit, residence status was checked and unreported SAE documented. Haematological and biochemical parameters were monitored on all participants; complete blood count at 1 month post dose 3 and creatinine, alanine aminotransferase [ALT] and bilirubin at 1 and 6½ months post dose 3.

### Immunogenicity Assessment

Hepatitis B surface antigen (HBsAg) status was determined in all participants prior to dose 1. Anti CS antibodies were measured prior to dose 1 and 30 days and 6½ months post dose 3 in Cohort 1 and anti-HBs antibodies at these same time points in Cohort 2. Indirect fluorescent antibody test (IFAT) were determined in both cohorts at screening.

### Efficacy Assessment

A health facility based morbidity surveillance system has been in operation since 1997¹³ and is currently established at Manhiça Health Center, and the Health Posts at Maragra and Ilha Josina. In all three facilities, project medical staff are available 24 hour a day to identify study participants through the personal ID card, and to ensure standardised documentation and appropriate medical management.

All children reporting fever within the preceding 24 hours or with a documented fever (axillary temperature ≧37.5° C.) had blood collected for determination of malaria parasites in duplicate thin and thick blood smears as well as a microcapillary tube for determination of the packed cell volume (PCV). Children with clinical conditions warranting hospitalisation were admitted to the Manhiça Health Center. On admission a more detailed clinical history and medical exam was performed and recorded on standardised forms by a physician. Results of laboratory investigations and the final diagnosis were recorded on discharge. Clinical management was carried out following standard national guidelines.

Active Detection of Infection (ADI) was carried out in cohort 2. Four weeks prior to the start of surveillance for malaria infection, asymptomatic parasitaemia was cleared presumptively with a combination of amodiaquine (10 mg/kg orally for 3 days) and SP (single oral dose sulfadoxine 25 mg/kg and pyrimethamine 1.25 mg/kg). The absence of parasitaemia was checked two weeks later and positives were treated with second line treatment (Co-Artem®) and excluded from further evaluation for ADI. Surveillance started 14 days after dose 3, and was carried out every two weeks for the following 2½ months and then monthly for a further two months (FIG. 1). At each visit, a field worker visited the child at home, completed a brief morbidity questionnaire and recorded the axillary temperature. If the child was afebrile, blood was collected by finger prick on to slides and filter paper. If the child was found to have fever or a history of fever, the child was accompanied to the Health Post were he/she was examined and blood slides collected. All children with a positive slide from the ADI were treated regardless of symptoms.

A cross sectional survey was carried 6½ months after dose 3 in both cohorts. During that visit axillary temperature and spleen size (Hackett's scale) were determined, and a blood slide prepared.

### Laboratory Methods

To determine parasite presence and density of P. falciparum asexual stages, Giemsa stained blood slides were read following standard quality-controlled procedures¹⁴. External validation was performed at the Hospital Clinic of Barcelona. Biochemical parameters were measured using a dry biochemistry photometer VITROS DT II (Orto Clinical Diagnostics, Johnson & Johnson Company, USA). Haematological tests were performed using a Sysmex KX-21N cell counter (Sysmex Corporation Kobe, Japan). Packed cell volume (PCV) was measured in heparinised rnicrocapillary tubes using a Hawksley haematocrit reader after centrifugation with a microhaematocrit centrifuge.

Antibodies specific for the circumsporozoite protein tandem repeat epitope were measured by a standard ELISA using plates absorbed with the recombinant antigen R32LR that contains the sequence [NVDP(NANP)15]2LR with a standard serum as a reference. The presence of HBsAg was determined by ELISA with a commercial kit (ETI-MAK-4 DIASOR®). Anti-HBsAg antibody levels were measured by ELISA with a commercial kit (AUSAB EIA from Abbott).For IFAT determination, 25 μl of test sera (two-fold serial dilutions up to 1/81920) were incubated with blood stage P. falciparum parasites fixed onto a slide. Positive reactions were revealed with FITC-labelled secondary antibody Evans Blue. The highest dilution giving positive fluorescence under a UV light microscope was scored.

### Definitions and Statistical Methods

The primary endpoint, evaluated in cohort 1, was time to the first clinical episode of symptomatic P. falciparum malaria. A clinical episode was defined as a child that presented to a health facility with an axillary temperature >37.5° C. and the presence of P. falciparum asexual parasitaemia above 2500 per μl. This case definition has been estimated to be 91% specific and 95% sensitive¹⁵. Secondary and tertiary endpoints included the estimation of vaccine efficacy for different definitions of clinical malaria and examining multiple episodes.

All hospital admissions were independently reviewed by two groups of clinicians in order to establish a final diagnosis, and discrepancies resolved in a consensus meeting prior to unblinding. Malaria requiring hospital admission was defined in a child with P. falciparum asexual parasitaemia where malaria was judged to be the sole cause of illness or a significant contributing factor. The case definition of severe malaria was derived from WHO's guide to clinical practice. All cases of severe malaria were required to have asexual P. falciparum parasitaemia and no other more probable cause of illness. The definition was a composite of severe malaria anaemia (PCV<15%), cerebral malaria (Blantyre coma score<2) and severe disease of other body systems: multiple seizures (at least 2 or more generalised convulsions in the previous 24 hours), prostration (defined as inability to sit unaided), hypoglycaemia (<2.2 mmol/dL), clinically suspected acidosis or circulatory collapse.

The According to Protocol (ATP) analysis of efficacy included subjects that met all eligibility criteria, completed the vaccination course and contributed to the efficacy surveillance. The time at risk was adjusted for absences from the study area and for antimalarial drug usage, except in estimates for all cause hospital admissions. For the analysis of multiple episodes of clinical malaria, a subject was not considered to be susceptible for 28 days after the previous episode.

For the time to first clinical malaria episode or malaria infection, vaccine efficacy was assessed using Cox regression models and was defined as 1 minus the hazard ratio. Vaccine efficacy was adjusted for predefined covariates of age, bed-net use, geographical area and distance from health centre. The proportional hazards assumption was investigated graphically, using a test based on the Schoenfeld residuals¹⁷ and time-dependent Cox models¹⁸. For multiple episodes of clinical malaria and hospital admissions, the group effect was assessed using Poisson regression models with normal random intercepts, including the time at risk as an off-set variable. Vaccine efficacy was defined as 1 minus rate ratio. The adjusted vaccine efficacy is reported throughout the text.

Further exploratory analyses included analyses on severe malaria and inpatient malaria, for which the difference in proportions of children with at least one episode were compared using the Fishers exact test. VE was calculated as 1 minus risk ratio, with exact 95% confidence interval¹⁹. The difference in anaemia prevalence (PCV<25%) and the proportion of positive parasite densities at Month 8½ were evaluated using the Fisher exact test. The effect of the treatment on hematocrit values and geometric mean of the positive densities were evaluated using the nonparametric Wilcoxon test.

Similar methodology was used in an intention to treat (ITT) analysis. Time at risk started from Dose 1, was not adjusted for absences from the study or drug usage, and the estimate of effect was not adjusted for covariates.

Anti-CS and anti-HBsAg antibody data were summarised by Geometric Mean Titres (GMTs) with 95% Cl. Seropositivity rates were calculated for anti-CS titres (defined as >0.5 EU/mL). Seroprotection rates were calculated for anti-HBs titres (defined as ≧10 mIU/mL). Analyses were performed using SAS²⁰ and STATA²¹.

### Results

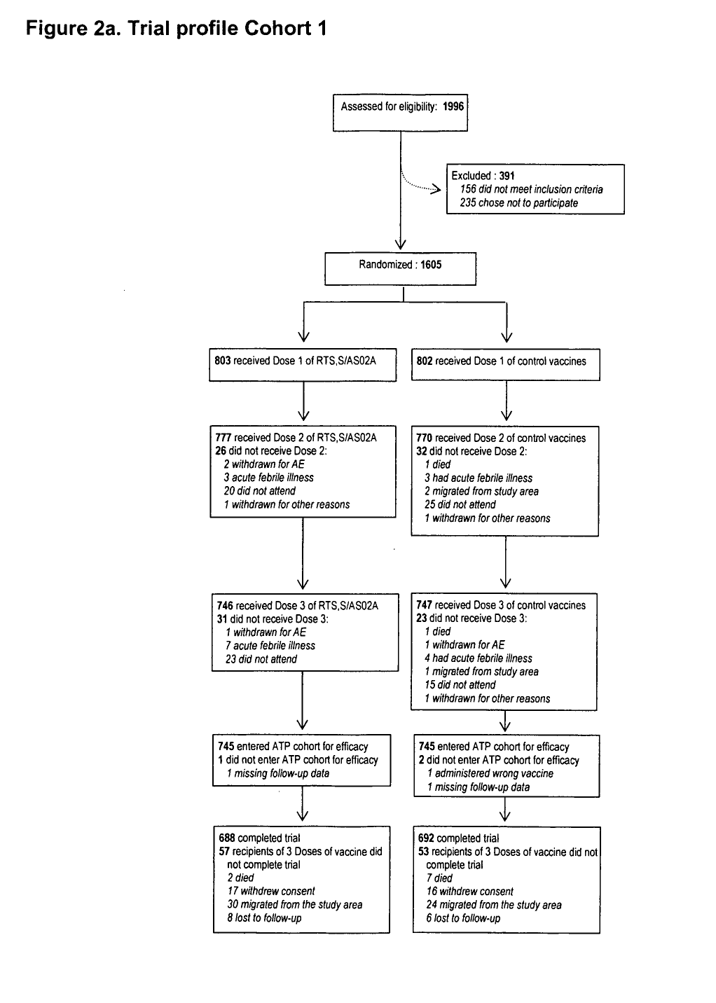

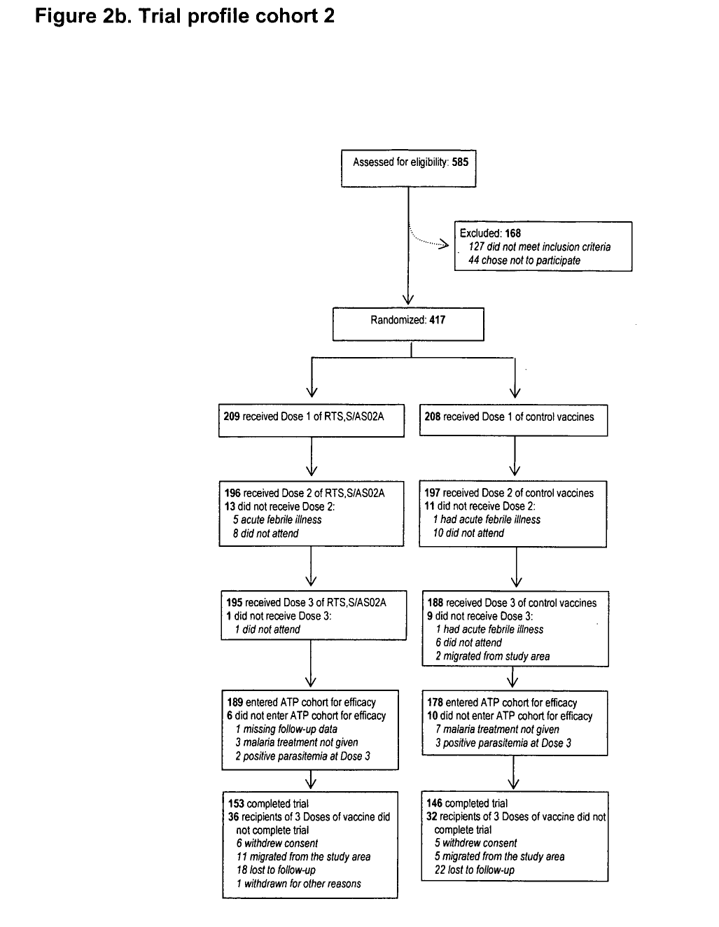

The trial profiles for cohorts 1 and 2 are shown in FIGS. 2a and 2b. Within each cohort, randomisation generated comparable groups of children (Table 1). All indicators suggest that malaria transmission intensity was higher in the study area of Cohort 2 than Cohort 1.

### Vaccine Safety

RTS,S/AS02A and control vaccines were safe and well tolerated; more than 92% of subjects in both groups received all three doses. Local and general solicited adverse events were of short duration, and mostly mild or moderate in intensity. Grade 3 local or general adverse events were uncommon and of short duration. In the RTS,S/AS02A and control groups, local injection site pain that limited arm motion occurred following 7 (0.2%) and 1 (0.03%) doses respectively, and injection site swelling>20 mm occurred following 224 (7.7%) and 14 (0.5%) doses respectively. General solicited adverse events (fever, irritability, drowsiness, anorexia) that prevented normal activities occurred following 55 (1.9%) and 23 (0.8%) of the doses in the RTS,S/AS02A and control groups, respectively. At least one unsolicited adverse event was reported by 653 (64.5%) subjects in the RTS,S/AS02A group and 597 (59.1%) subjects in the control group. Safety laboratory values remained essentially unchanged from baseline over the course of the trial.

There were 429 reported SAEs: 180 [17.8%] in the RTS,S/AS02A group vs 249 [24.7%] in the control group. There were 15 deaths during the study: 5 [0.6%] in the RTS,S/AS02A group and 10 [1.2%] in the control group. Four deaths had malaria as a significant contributing factor, and all four were in the control group. No serious adverse event or death was judged to be related to vaccination.

### Immunogenicity

Pre vaccination anti-CS antibody titres were low in the study children. The vaccine was immunogenic, inducing high antibody levels after dose 3, decaying over 6 months to about ¼ of the initial level, but remaining well above baseline values. Antibody levels in the control group remained low throughout the follow up period. The vaccine also induced high levels of anti-HBsAg antibodies (greater than 97% seroprotection) (Table 2). For both CS and HBsAg, the immunogenicity of the vaccine was greater in children below 24 months of age.

### Vaccine Efficacy

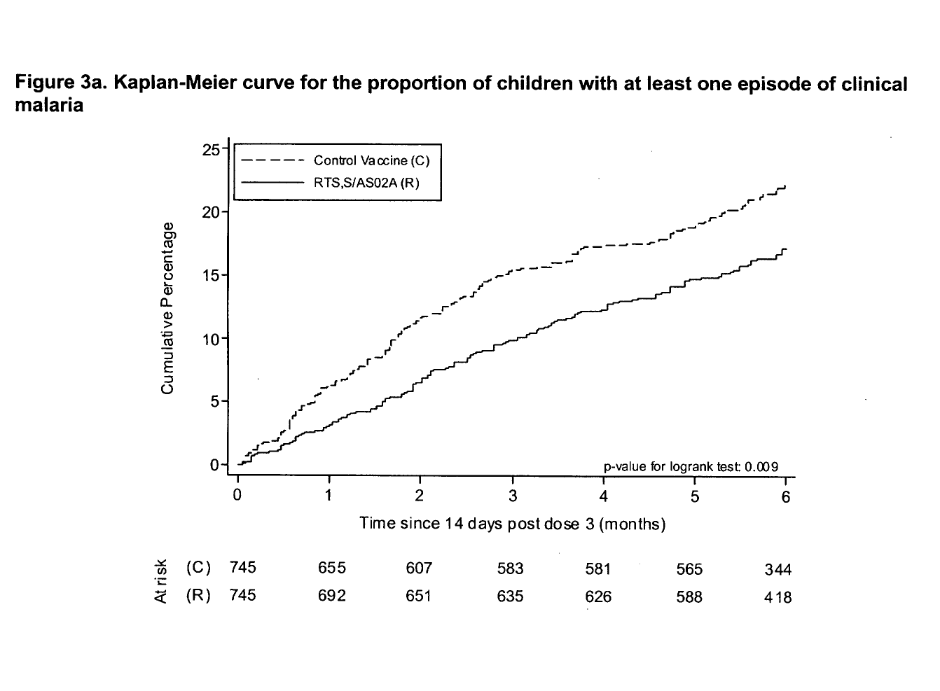

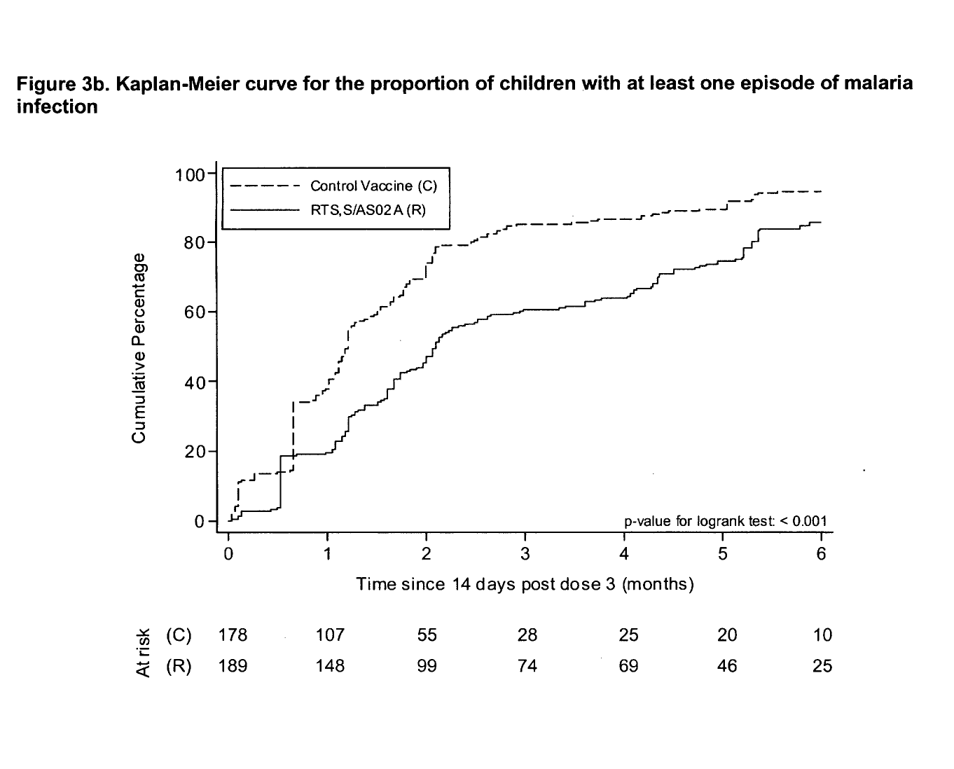

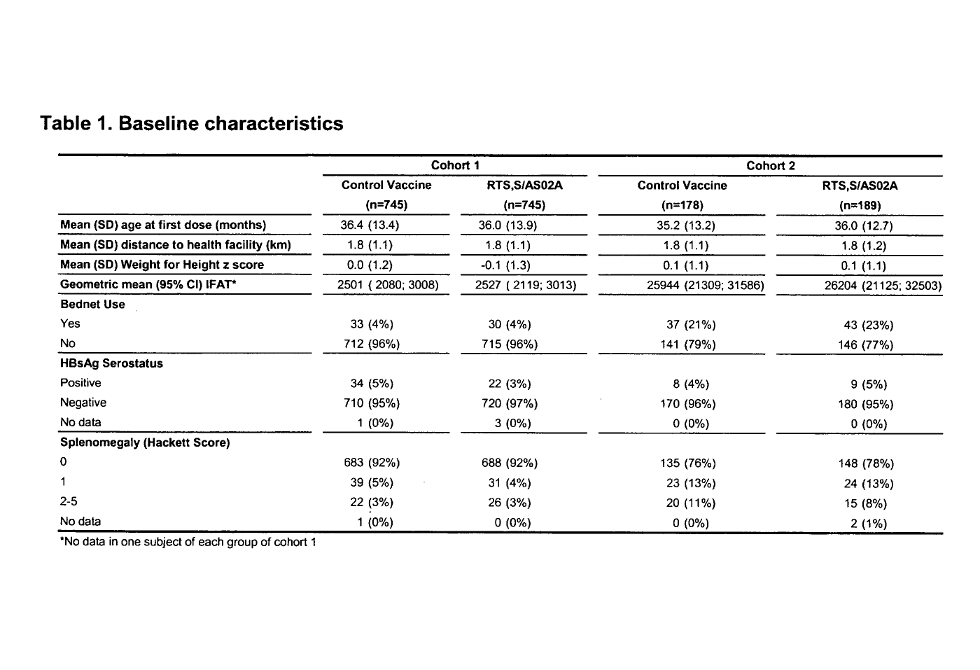

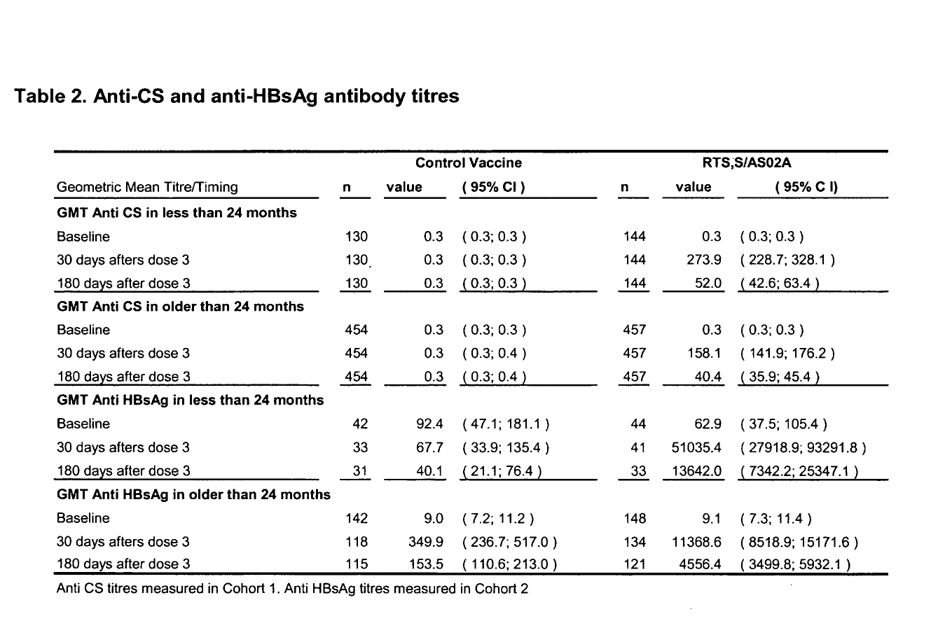

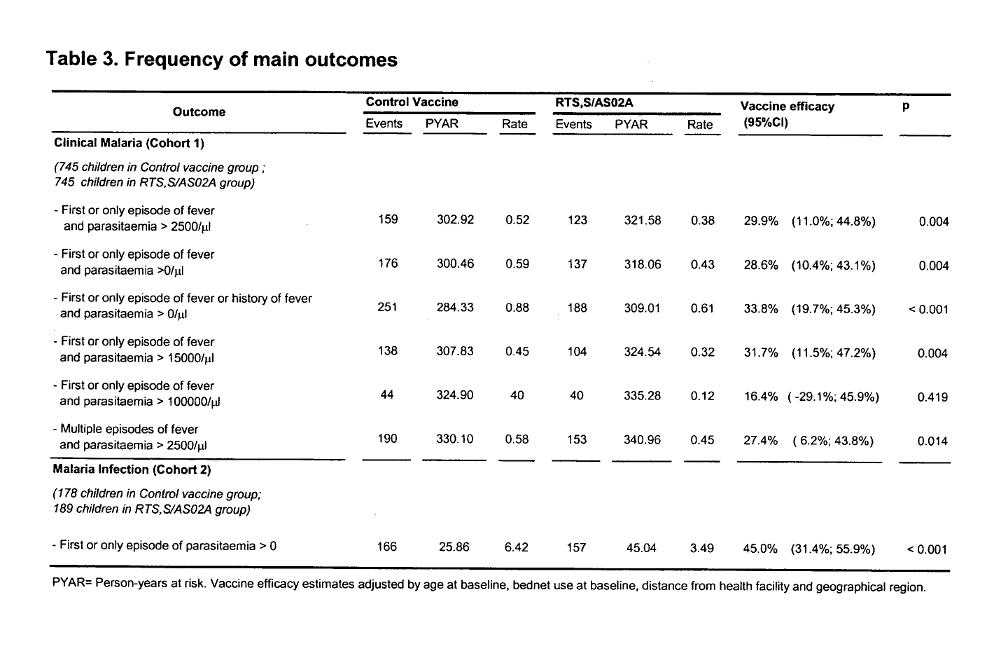

In the ATP analysis performed in cohort 1, there were 282 children with first or only clinical episodes meeting the primary case definition (123 in the RTS,S/AS02A group and 159 in the control group), yielding a crude vaccine efficacy estimate of 26.9% (95%CI: 7.4%-42.2%; p=0.009) and an adjusted estimate of 29.9% (95% CI: 11 %-44.8%; p=0.004) (FIG. 3a and Table 3). The density of asexual stage parasites among children with a first episode of clinical malaria was not affected by vaccination as the geometric mean densities at time of presentation were 43 522/μL and 41 867/μL for the RTS,S/AS02A and control groups, respectively (p=0.915).

There was no evidence of waning efficacy as defined in the primary endpoint over the six month observation period when analysed using different methods (test for the proportionality of the hazards using Schoenfeld residuals [p=0.139]). Consistent with these data, at the cross-sectional survey 6½ months after Dose 3, the prevalence of parasitaemia among RTS,S/AS02A recipients was 37% lower (11.9% in RTS,S/AS02A vs 18.9% in controls, p<0.001). Parasite densities in these children were similar between RTS,S recipients and controls (geometric mean density 2271 vs 2513; p=0.699).

Few children experienced more than one episode and the vaccine efficacy for this endpoint was VE=27.4% [95% CI: 6.2%-43.8%; p=0.014]). The VE estimate did not significantly change for different case definitions based on parasite density cut offs (Table 3). An ITT analysis of time to clinical disease starting from dose 1 yielded VE of 30.2% (95% CI: 14.4%-43.0%; p<0.001). In the ATP analysis, there were 26 incident episodes of anaemia (PCV<25%) in the RTS,S/AS02A group and 36 in the control group (VE=28.2% [95% CI: −19.6%-56.9%; p=0.203]). The prevalence of anaemia at month 8½ was 0.29% in the control group vs 0.44% in the vaccine group, p=0.686.

In the RTS,S/AS02A group there were 11 children who had at least one episode of severe malaria while in the control group there were 26 children (VE=57.7% [95% CI: 16.2%-80.6%; p=0.019]). In the RTS,S/AS02A group, there were 42 children with malaria that required hospital admission versus 62 in the control group (VE=32.3% [95% CI: 1.3%-53.9%; p=0.053]). There were similar numbers of all cause hospital admissions between the two groups(79 vs 90; VE=14.4% [95% CI: −19.7%-38.8%; p=0.362]).

Evaluation of the efficacy of the vaccine in reducing time to first infection was determined in Cohort 2. There were 323 children with first or only episodes of asexual P. falciparum parasitaemia (157 in the RTS,S/AS02A group and 166 in the control group) yielding a VE estimate of 45% (95% CI: 31.4%-55.9%; p<0.001) (FIG. 3b and Table 3). The mean density of asexual stage parasites at the time of first infection were similar for the control and RTS,S/AS02A groups (3950/μL vs 3016/μL, p=0.354). Using the same methods as those used to assess persistence of efficacy for Cohort 1, the model with the best fit suggested waning in the efficacy of the vaccine over time, that stabilised at about 40%. The prevalence of asexual P. falciparum parasitaemia at the end of follow-up was significantly lower in the RTS,S/AS02A than in the control group (52.3% vs. 65.8%; p=0.019) respectively. The prevalence of anaemia at month 8½ was 2.7% in the control group and 0.0% in the RTS,S/AS02A group (p=0.056).

There was no evidence of an interaction between age and vaccine efficacy, suggesting that efficacy did not significantly change with increasing age. We did however carry out further exploratory subgroup analysis to estimate vaccine efficacy in the younger age groups that carry the brunt of malaria disease. Among children<24 months of age at time of dose 1, there were 3 cases of severe malaria among the recipients of RTS,S/AS02A (N=173) while there were 13 cases among the recipients of control vaccines (N=173) (VE=76.9% [95% CI: 27.0%-96.9%; p=0.018]). The incidence of first or only episodes of clinical malaria was similarly analysed. There were 31 and 47 episodes of malaria in younger children, yielding incidence rates of 0.41 and 0.70 episodes PYAR in the RTS,S/AS02A and control groups respectively (VE=46.7% [95% CI: 14.8%-66.7%; p=0.009]). VE against new infections was similar in the older and younger age groups (44.0% versus 46.5%).

The relationship between CS titres and malaria protection was evaluated in Cohort 1. The hazard ratio per 10-fold increase in CS titre was 0.94 (95% CI: 0.66-1.33; p=0.708); the hazard ratio for the comparison of subjects in the higher tertile of CS response vs subjects in the lower tertile of CS response was 1.38 (95% CI: CI 0.89-2.12; p=0.150).

### Discussion

RTS,S/AS02A is the first subunit vaccine to confer protection in young African children against both infection and a spectrum of clinical illness caused by P. falciparum. The results show that a vaccine based on a single pre erythrocytic antigen that induces partial protection against infection can reduce morbidity, even in the absence of a blood stage component.

In young African children, RTS,S/AS02A was well tolerated and its reactogenicity profile was similar to that observed in previous paediatric trials of this vaccine. Local and general symptoms were more common than in the control vaccine group, but did not lead to withdrawals of subjects. The vaccine was safe; children who received RTS,S/AS02A experienced fewer all-cause serious adverse events, hospitalisations and severe complications from malaria, than did those in the control group. As has been seen in other intervention trials, the mortality rate among our study participants was lower than historical background mortality rates in this population⁹.

Despite high levels of exposure to P. falciparum sporozoites, naturally occurring anti CS antibody levels in this population were low. The vaccine was highly immunogenic, especially in children less than 24 months. Antibody levels decayed by approximately 75% over 6 months, but at the end of the follow up period, they were still well above pre immunisation levels. Among RTS,S/AS02A recipients, we failed to detect an association between the level of anti CS antibodies and the risk of malaria. However, the high titres achieved by nearly all vaccine recipients and the possibility that a relatively low threshold protective level of immunity may exist potentially constrained this analysis. Also, the vaccine is known to induce cell-mediated responses believed to be involved in protection that were not measured in this study²².

The vaccine's efficacy against infection is consistent with the known ability of this pre erythrocytic vaccine to neutralise sporozoites and limit the number of infected hepatocytes or liver stage merozoites that enter the blood stream⁵. The results also show remarkable consistency between protection against infection, and protection against mild uncomplicated disease, malaria hospital admissions and severe malaria. While there seems to be a trend suggesting that efficacy is higher in the younger children and for the more severe endpoints, confidence intervals for the different endpoints overlap, and observed differences may be due to chance. The observed protection against different endpoints suggests that the more easily measured infection endpoint may serve as a surrogate for vaccine efficacy against clinical disease.

We were surprised not to see a significant difference in cases of anaemia. Although the trend was for lower number of cases to occur in the recipients of RTS,S/AS02A vaccine, the rates of malaria anaemia during the study were much lower than expected and this limited the ability to detect statistically significant vaccine efficacy for this endpoint. Intense prompting of the mothers or guardians to take their children to health facilities early on in the disease process may have ensured prompt treatment of the malaria cases and reduced the incidence of anaemia. In addition, Mozambique recently switched to a more effective first line treatment for malaria and children in the trial who received these drugs had more rapid clearing of parasites, fewer recrudescence and therefore shorter duration of infections. Each of these interventions may have had an impact on the observed incidence of anaemia.

The statistical methods we used to detect waning efficacy suggested that there was continued vaccine efficacy against both new infections and clinical disease throughout the observation period, and at the last cross-sectional survey there was a significant difference in the prevalence of infection. This is in sharp contrast from trials in malaria naïe volunteers or Gambian adults which suggested that vaccine efficacy was short lived ^{6,23}. There are several possible explanations for these apparently conflicting results. Firstly, the vaccine was much more immunogenic in this study population than it was in adults and sustained immune responses may have resulted in persistent protective efficacy. Secondly, the higher level of sporozoite exposure that occurred during this trial may have resulted in natural boosting of protective immune responses not revealed by antibody measurements. The study population remains under surveillance to monitor both long term safety and the duration of vaccine efficacy.

One of the most remarkable findings of this trial is the documented efficacy against severe malaria of 58%, and the suggestion that it may be higher in younger children. Although the definition of severe malaria is a matter of continuous discussion, there is little doubt that classification of children according to the WHO-based definition identifies children who are very sick and at high risk of dying.

### REFERENCES

1. Hay S I, Guerra C A, Tatem A J, Noor A M, Snow R W. The global distribution and population at risk of malaria: past, present, and future. The Lancet Infectious Diseases 2004;4(6):327-336.

2. Breman J G, Alilio M S, Mills A. The intolerable burden of malaria: what's new, what's needed. Am J Trop Med Hyg 2004;71(2_suppl):0-i-.

3. Klausner R, Alonso P. An attack on all fronts. Nature 2004;430(7002):930-1.

4. Ballou W R, Arevalo-Herrera M, Carucci D, Richie T L, Corradin G, Diggs C, et al. Update on the clinical development of candidate malaria vaccines. Am J Trop Med Hyg 2004;71 (2_suppl):239-247.

5. Stoute J, Slaoui M, Heppner D, Momin P, Kester K, Desmons P, et al. A preliminary evaluation of a recombinant circumsporozoite protein vaccine against Plasmodium falciparum malaria. RTS,S Malaria Vaccine Evaluation Group. N Engl J Med 1997;336(2):86-91.

6. Bojang K A, Milligan P J M, Pinder M, Vigneron L, Alloueche A, Kester K E, et al. Efficacy of RTS,S/AS02 malaria vaccine against Plasmodium falciparum infection in semi-immune adult men in The Gambia: a randomised trial. The Lancet 2001;358(9297):1927-1934.

7. Bojang K A,, Olodude F, Pinder M, Ofori-Anyinam 0, Vigneron L, Fitzpatrick S, Njie F, Kassanga A, Leach A, Milman J, Rabinovich R, McAdam K P W J, Kester K E, Heppner D G, Cohen J D, Tornieporth N, and Milligan P J M. Safety and immunogenicity of RTS,S/AS02A candidate malaria vaccine in Gambian children. Vaccine submitted.

8. Macete E, Aponte J J, Guinovart C, Sacarlal J, Mandomando I, Espasa M, et al. Safety, reactogenicity and immunogenicty of the RTS,S/AS02A candidate malaria vaccine in children aged 1 to 4 years in Mozambique. Vaccine submitted.

9. Alonso P, Sáute F, Aponte J, Gómez-Olivé F, Nhacolo A, Thomson R, et al. Manhiça DSS, Mozambique. In: INDEPTH, ed. Population and Health in Developing Countries. Ottawa: International Development Research Centre, 2001: 189-195.

10. Dame J B, Williams J L, McCutchan T F, Weber J L, Wirtz R A, Hockmeyer W T, Maloy W L, Haynes J D, Schneider I, Roberts D, et al. Structure of the gene encoding the immunodominant surface antigen on the sporozoite of the human malaria parasite Plasmodium falciparum. Science. 1984;225:593-9.

11. Young J F, Hockmeyer W T, Gross M, Ballou W R, Wirtz R A, Trosper J H, Beaudoin R L, Hollingdale M R, Miller L H, Diggs C L, et al. Expression of Plasmodium falciparum circumsporozoite proteins in Escherichia coli for potential use in a human malaria vaccine. Science 1985;228:958-62.

12. Doherty J, Pinder M, Tornieporth N, Carton C, Vigneron L, Milligan P, et al. A phase I safety and immunogenicity trial with the candidate malaria vaccine RTS,S/SBAS2 in semi-immune adults in The Gambia. Am J Trop Med Hyg 1999;61(6):865-868.

13. Loscertales M P, Roca A, Ventura P, Abascassamo F, Dos Santos F, Sitaube M, et al. Epidemiology and clinical presentation of respiratory syncytial virus infection in a rural area of southern Mozambique. Pediatr Infect Dis J 2002;21:148-155.

14. Alonso P, Smith T, Schellenberg J, Masanja H, Mwankusye S, Urassa H, et al. Randomised trial of efficacy of SPf66 vaccine against Plasmodium falciparum malaria in children in southern Tanzania. The Lancet 1994;344:1175-81.

15. Saute F, Aponte J, Almeda J, Ascaso C, Abellana R, Vaz N, et al. Malaria in southern Mozambique: malariometric indicators and malaria case definition in Manhiça district. in press.

16. World Health Organization. Management of severe malaria, a practical handbook. Second edition, 2000. http://mosquito.who.int/docs/hbsm.pdf

17. Therneau T M, Grambsch P M. Modeling Survival Data: Extending the Cox Model. New York: Springer, 2000.

18. Hess K R. Graphical methods for assessing violations of the proportional hazards assumption in Cox regression. Stat Med 1995;14(15):1707-23.

19. Cytel Software Corporation. StatXact PROCs for SAS Users (version 6). Cambridge, Mass., USA.

20. SAS Institue Inc. SAS software (version 8). Cary, N.C., USA.

21. Stata Corporation. Stata Statistical Software (Release 8.0). College Station, Tex., USA 2003.

22. Sun P, Schwenk R, White K, Stoute J A, Cohen J, Ballou W R, Voss G, Kester K E, Heppner D G, Krzych U. Protective immunity induced with malaria vaccine, RTS,S, is linked to Plasmodium falciparum circumsporozoite protein-specific CD4(+) and CD8(+) T cells producing IFN-gamma. J Immunol. 2003 Dec 15; 171(12): 6961-7.

23. Stoute, J A, Kester K E, Krzych U, Wellde B T, Hall T, White K, Glenn G, Ockenhouse C F, Garçon N, Schwenk R, Lanar D E, Momin P, Golenda C, Slaoui M, Wortmann G, Cohen J, Ballou W R. Long Term Efficacy and Immune Responses Following Immunization with the RTS,S Malaria Vaccine. J Infect Dis 178:1139-44, 1998.

1.-15. (canceled)

16. A method for eliciting an immune response that protects against severe malarial disease comprising:
administering to a subject a composition comprising a Plasmodium circumsporozoite protein (CS) antigen in combination with a pharmaceutically acceptable adjuvant or carrier.

17. The method of claim 16, wherein the subject is a child under 5 years of age.

18. The method of claim 16, wherein the subject is a child between 1 and 4 years of age.

19. The method of claim 16, wherein the antigen is fused to the surface antigen from hepatitis B (HBsAg).

20. The method of claim 16, wherein the CS protein or fragment is in the form of a hybrid protein comprising substantially all the C-terminal portion of the CS protein of Plasmodium, four or more tandem repeats of the CS protein immunodominant region, and the surface antigen from hepatitis B (HBsAg).

21. The method of claim 20, wherein the hybrid protein comprises a sequence of CS protein of P. falciparum substantially as corresponding to amino acids 207-395 of P. falciparum NF54 strain 3D7 clone CS protein fused in frame via a linear linker to the N-terminal of HBsAg.

22. The method of claim 20, wherein the hybrid protein is RTS.

23. The method of claim 22, wherein the RTS is in the form of mixed particles RTS,S.

25. The method of claim 23, wherein the amount of RTS,S is 25 μg per dose.

26. The method of claim 16, wherein the composition further comprises an adjuvant, which adjuvant is a preferential stimulator of a Th1 cell response.

27. The method of claim 26, wherein the adjuvant comprises 3D-MPL, QS21 or a combination of 3D-MPL and QS21.

28. The method of claim 27, wherein the adjuvant further comprises an oil in water emulsion.

29. The method of claim 28, wherein the adjuvant further comprises liposomes.

30. The method of claim 16, comprising administering a plurality of doses of the composition comprising the Plasmodium antigen that is expressed at the pre-erythrocytic stage.
